(19) United States
(12) Patent Application Publication
(10) Pub. No.: US 2013/0096552 A1
(43) Pub. Date: Apr. 18, 2013

(54) Hydrodissection Material with Reduced Migration

(71) Applicants: Christopher L. Brace, Madison, WI (US); James L. Hinshaw, Middleton, WI (US); Meghan G. Lubner, Madison, WI (US); Anthony J. Sprangers, Appleton, WI (US); Alexander D. Johnson, Madison, WI (US); Patrick R. Cassidy, Madison, WI (US); Sean R. Heyman, Madison, WI (US)

(72) Inventors: Christopher L. Brace, Madison, WI (US); James L. Hinshaw, Middleton, WI (US); Meghan G. Lubner, Madison, WI (US); Anthony J. Sprangers, Appleton, WI (US); Alexander D. Johnson, Madison, WI (US); Patrick R. Cassidy, Madison, WI (US); Sean R. Heyman, Madison, WI (US)

(21) Appl. No.: 13/274,036

(22) Filed: Oct. 14, 2011

(51) Int. Cl. A61B 18/18

(52) U.S. Cl. 606/41

(57) ABSTRACT

A hydrodissection material provides a flowable biocompatible material that increases in viscosity in situ to reduce material migration during an ablation procedure. One embodiment provides a material that increases in viscosity at normal body temperatures to permit injection using a standard hypodermic needle.

### STATEMENT REGARDING FEDERALLY SPONSORED RESEARCH OR DEVELOPMENT

--

### CROSS REFERENCE TO RELATED APPLICATION

--

### BACKGROUND OF THE INVENTION

The present invention relates the treatment of tumors by ablation of tissue and in particular to a “hydrodissection” material protecting adjacent tissues during the ablative or similar procedure.

Tissue ablation has become increasingly accepted for treatment of malignant tumors of the heart, lungs, liver, and kidneys. Such ablation techniques include: radiofrequency (RF) ablation, cryoablation, microwave ablation, laser ablation, ethanol ablation, and chemoembolization.

Radiofrequency ablation may employ one or more needle-like electrodes inserted into the tumor to introduce electrical radiofrequency current flow therethrough either among electrodes (bi-polar) or from the needle-like electrodes to a large area ground pad on the patient's skin. Ohmic heating of the tissue can destroy tumor cells within an ablation zone of about 3 cm from a single electrode, while an umbrella shaped electrode has a slightly larger ablation zone.

The three main methods of RF ablation are surgical, laparoscopic, and percutaneous. Use of surgical methods is the most invasive and involves opening the patient for precise probe placement and requires the use of general anesthesia. In the laparoscopic method, an incision is made in the skin, through which a laparoscope is inserted. The laparoscope is then used to accurately place the RF electrode(s). Percutaneous RF ablation inserts the RF electrode through the skin using imaging guidance, such as ultrasound, computed tomography (CT), and magnetic resonance imaging (MRI). This approach can often be performed with lighter sedation, and is associated with fewer and less severe complications, lower monetary cost, and faster recovery than surgical approaches.

Cryoablation uses extremely cold temperatures to kill tumor tissue. Cryoablation can also be performed surgically, laparoscopically, or percutaneously using a cryoprobe. The cryoprobe circulates liquid nitrogen or argon internally to rapidly cool the surrounding tissue to cytotoxic levels. Multiple cryoprobes can work simultaneously in concert to treat larger tumor volumes. The iceball created during cryoablation can be visualized using medical imaging equipment.

Most ablation techniques do not inherently differentiate between tumor and non-tumor tissue, so the medical personnel must keep the treatment as focal as possible to treat the tumor with adequate margin but avoid damage to vulnerable normal tissues. Such tissues frequently include the diaphragm, stomach, bowel, body wall or bladder. To help with this, a layer of protective fluid may be injected into the patient around the target area in a process known as hydrodissection. This fluid layer separates the target and surrounding tissue, creating a barrier to protect the adjacent vulnerable non-target tissue from the potential damaging effects of the ablation procedure. There are three current options used for hydrodissection: normal saline, 5% dextrose in water (D5W), and carbon dioxide (CO₂).

Normal saline is a commonly available salt solution adjusted to be isotonic to body tissue (typically 0.91% NaCl) and may be injected percutaneously near the site of ablation. Since normal saline is composed of mostly water, it has a high specific heat and shields non-tumor tissue well from extreme temperature changes. Unfortunately, the intraperitoneal (IP) pressure of the body cavity can often cause migration of the saline away from the target tissue and because of this, large amounts (>1 L) may be necessary to obtain adequate tissue displacement (1-2 cm). Normal saline is also a good electrical conductor that is often used to enhance RF ablations; therefore, it is not a good option for hydrodissection during RF ablation procedures.

Carbon dioxide may be administered in two ways: via a gas-filled balloon or via insufflation (injection of gas into the body cavity). Balloons are more invasive, more expensive and more technically challenging to place than direct fluid injection. Direct injection of CO₂ can also be difficult to control within the peritoneal cavity. As a result, the procedure requires the use of several gas bags or large amounts of CO₂ (>1 L). And while CO₂ is an efficient thermal and electrical insulator, it is a poor acoustic medium, making it incompatible with transcutaneous ultrasonic imaging.

The most commonly used hydrodissection fluid, D5W, is a sterilized isotonic solution of dextrose and water that is commonly used as intravenous fluid. It is both cheap and plentiful in the hospital environment and can be easily introduced to the target area by percutaneous injection. Unlike saline, however, it is not electrically conductive. This reduces unwanted tissue damage by as much as 35% compared to saline which is electrically conductive. As with saline solutions, large volumes (>1 L) of D5W may be required to adequately protect tissue due to migration of the solution within, or absorption by, the body cavity.

### SUMMARY OF THE INVENTION

The present invention provides a method and apparatus concerning a hydrodissection material having adjustable viscosity so as to permit introduction of the material, for example, percutaneously in a low viscosity state, and then, once the material is in place near an ablation site, conversion of the material to a high viscosity state by heat of the body or introduction of a trigger substance. The higher viscosity reduces migration of the hydrodissection material and hence the volume of material that must be introduced into the body.

Specifically, in one aspect, the invention provides a method of treating tumors including the steps of introducing a biocompatible gelable material in a liquid phase between a first and second tissue region to separate the regions and converting the liquid phase gel to a gel phase having a substantially greater viscosity to resist migration from between the first and second tissue regions. Once the gel is in place, a destructive agent (e.g. heat, cold, radiation, or chemicals) may be applied to the first tissue to destroy a tumor portion thereof. The separation of the first and second tissue is selected to protect the second tissue from the destruction of the first tissue.

It is thus a feature of at least one embodiment of the invention to provide a hydrodissection material that better resists migration in the body thus providing more stable and consistent protection and reducing fluid loading to the patient.

The gelable liquid may change from the liquid phase to the gel phase as a function of temperature and wherein the gel phase occurs at substantially normal body temperature which causes a temperature induced phase change of the gelable liquid; and the step of converting the gelable liquid to a gel phase may be invoked by a temperature change of the gelable liquid.

It is thus a feature of at least one embodiment of the invention to provide a simple, controllable method of increasing the viscosity of the material without the need for careful timing or additional gelling agents.

The method may include the step of cooling the gelable liquid to substantially no greater than room temperature at a time of introduction.

It is thus a feature of at least one embodiment of the invention to provide a material that may be introduced into the body below body temperature so as to reduce tissue damage in contrast to materials requiring elevated temperature. It is another feature of at least one embodiment of the invention, when the cooling is substantially to room temperature, to provide a material that may be prepared at room temperature without the need for refrigeration or the like.

Alternatively, the gelable liquid may change from the liquid phase to the gel phase in the presence of a gelling trigger material; and the step of converting the gelable liquid to a gel phase may be provided by the introduction of the gelling trigger material into contact with the introduced gelable liquid.

It is thus a feature of at least one embodiment of the invention to provide a material that may be relatively insensitive to temperature conditions.

The treatment may be selected from a group consisting of, but not limited to: cryoablation, microwave ablation, radiofrequency ablation, laser ablation, ethanol ablation, interstitial or external ultrasound ablation, internal or external radiotherapy, and chemoembolization. Further the first and second tissues may be selected from the tissue pair groups of: liver/diaphragm, liver/bowel, liver/stomach, kidney/bowel, and gall bladder/liver.

It is thus a feature of at least one embodiment of the invention to provide a system broadly applicable to a range of ablation types and situations.

The gelable liquid may include a contrast agent and the method may further include the step of monitoring the introduction of the contrast agent with an image modality sensitive to the contrast agent.

It is thus a feature of at least one embodiment of the invention to permit imaging of the hydrodissection process.

The step of introducing the gelable liquid may include injecting the gelable liquid through a hypodermic needle having an inner diameter no greater than one millimeter.

It is thus a feature of at least one embodiment of the invention to provide a material that may be used percutaneously.

The gelable liquid may have a liquid molecular weight of less than 13 KDa.

It is thus a feature of at least one embodiment of the invention to provide a material that may be bio-absorbed and discharged from the body after the ablation procedure.

The gelable liquid may be a micelle-forming polymer.

It is thus a feature of at least one embodiment of the invention to provide a material that thickens with increased temperature.

The gelable liquid may be a solution of a poloxamer, for example, a solution of Poloxamer-407 and water having a weight-based dilution ratio of between 14 and 18 percent Poloxamer-407.

It is thus a feature of at least one embodiment of the invention to make use of the commonly available and well-characterized material.

These particular objects and advantages may apply to only some embodiments falling within the claims and thus do not define the scope of the invention.

### BRIEF DESCRIPTION OF THE DRAWINGS

FIG. 1 is a view, in phantom, of an example hydrodissection procedure employing the method and material of the present invention for treating a liver tumor adjacent to a patient's diaphragm by thermal ablation;

FIG. 2 is a fragmentary enlarged cross-section of an ablation area of FIG. 1 showing a separating layer of hydrodissection material;

FIG. 3 is a figure similar to that of FIG. 2 showing the hydrodissection process with an alternative two-part hydrodissection material;

FIG. 4 is a chemical formula for a solute of one hydrodissection material (Poloxamer-407) having a hydrophobic center block and hydrophilic ends;

FIG. 5 is a simplified representation of the gelling process in poloxamer molecules form micelles which then organize into micelle structures in a gelling process with temperature rise;

FIG. 6 is a simplified diagram showing increase in viscosity of one hydrodissection material with temperature increase; and

FIG. 7 is a simplified diagram showing change in gelation temperature as a function of concentration for Poloxamer-407.

### DETAILED DESCRIPTION OF THE PREFERRED EMBODIMENT

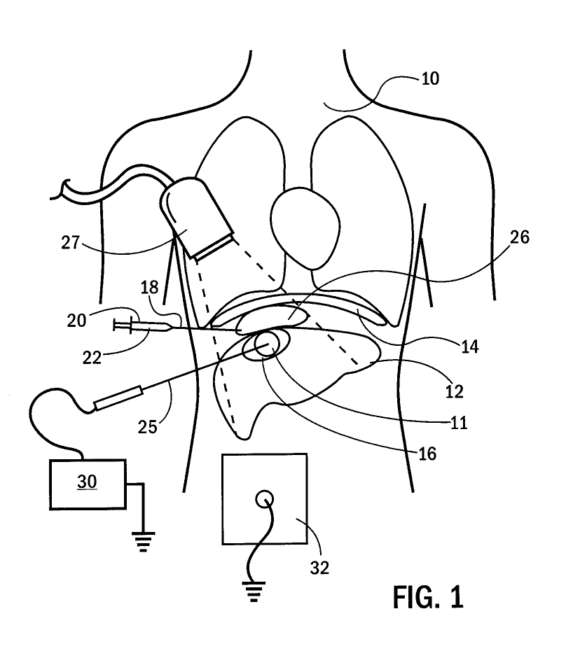

Referring now to FIG. 1, in one example, the present invention may be used in a patient 10 during ablation of a tumor 11 in a patient's liver 12 adjacent to the patient's diaphragm 14. In this situation, it is important that an ablation region 16 around the tumor 11 not extend into the diaphragm 14 in order to protect the diaphragm 14 from damage and prevent adhesion between the diaphragm and liver.

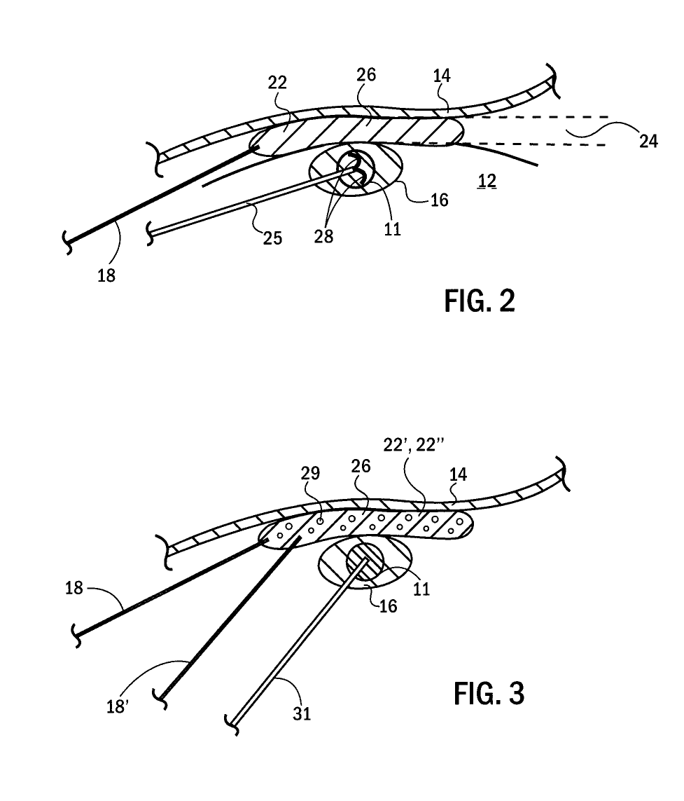

Referring also to FIG. 2, in a first embodiment of the present invention, a hypodermic needle 18, for example, of 17 gauge or higher (approximately 1 millimeter internal diameter or smaller with increasing gauge), and preferably 19 gauge, may be attached to a syringe 20 holding a gelable hydrodissection material 22 of the present invention. At this time the gelable hydrodissection material 22 is in a liquid state suitable for injection through the hypodermic needle 18 and may have a kinetic viscosity of less than 18 centiStokes and typically on the order of 10-30 centiStokes.

A distal end of the needle 18 may be inserted percutaneously to a position between the tumor 11 and the diaphragm 14, and the gelable hydrodissection material 22 injected in between the liver 12 and the diaphragm 14 to form a separating layer 26. The imposition of the gelable hydrodissection material 22 as the separating layer 26 physically causes separation of the liver 12 and diaphragm 14 by separation distance 24 for example 5 mm or more and preferably 1-2 centimeters and may provide both electrical and thermal separation through electrical and thermal resistance of the gelable hydrodissection material 22 and thermal capacitance of the gelable hydrodissection material 22.

An elevation in temperature of the gelable hydrodissection material 22 to body temperature once in place within the body of the patient 10 may cause it to gel increasing its viscosity to above 18-30 centiStokes (typically to a solid gel with infinite viscosity), improving its ability to maintain separation between the liver 12 and the diaphragm 14 and preventing its migration within the patient 10.

During the injection of the gelable hydrodissection material 22, the distal end of the needle 18 may be drawn along the interface between the liver 12 and the diaphragm 14 under the guidance of ultrasound images obtained via ultrasound imaging probe 27 positioned appropriately.

Once the separating layer 26 is in place, the ablation process may proceed by the percutaneous insertion of an ablation electrode 25, for example, having distal extendable umbrella prongs 28, into the tumor 11. This process may also be guided by ultrasonic imaging through ultrasound imaging probe 27.

The ablation electrode 25 may be connected to a radiofrequency electrical power source 30 having an electrical return connected through a large area ground pad 32 placed elsewhere on the skin of the patient 10. Monopolar electrical current will then cause the formation of the ablation region 16 about the distal end of the ablation electrode 25 whose growth toward the diaphragm 14 is substantially blocked by the separating layer 26 which provides for both electrical and thermal blockage protecting the diaphragm 14. The invention may also be used with bipolar current flows between one or more ablation electrodes 25 where the separating layer 26 provides thermal isolation and constrain fringing current flow.

It will be understood that this procedure may be used between any two separable tissue structures one of which is to be treated by ablation and the other of which is to be protected or shielded for example the interfaces between liver/diaphragm, liver/bowel, liver/stomach, kidney/bowel, etc. In addition it will be understood that the technique may be used for a variety of different ablation processes including cryoablation, microwave ablation, radiofrequency ablation, laser ablation, ethanol ablation, and chemoembolization.

Referring now to FIG. 3, in an alternative embodiment, a first and second hypodermic needle 18 and 18′ may be used to deposit a separating layer 26 formed of the intermixing of two different hydrodissection materials 22′ and 22″ through each of the hypodermic needles 18 and 18′. Each of the different hydrodissection materials 22′ and 22″ may have a liquid state prior to mixing within the body of the patient 10 whereupon chemical interaction between materials from each of the hypodermic needles 18 and 18′ converts the liquid hydrodissection material 22′ and 22″ from the hypodermic needles 18 into a gel state.

One or both of the hydrodissection material 22′ and 22″ may include a contrast agent 29 tailored for the particular image modality that may be used for guiding the ablation process. For example, for computed tomography, the contrast agent 29 may for example be ½ to 3 percent weight to volume of isohexyl and preferably 1.5 percent. Other contrast agents believed to be compatible with this process include ultrasound blocking microspheres; x-ray blocking iodine, and MRI sensitive gadolinium. An optical contrast agent such as India ink may also be added to permit visual identification of the separation layer 26 and any of the hydrodissection materials 22, 22′ and 22″ at later surgical excision.

Once the separating layer 26 is formed and gelled, a similar ablation technique may be used to form an ablation region about the tumor 11. In this example, a cryoablation probe 31 may be used to form the ablation region 16 about the tumor 11 by freezing the tissue in the ablation region 16. In this case the electrical blocking abilities of the separating layer 26 are not critical but rather the thermal blocking abilities of that latter.

Upon completion of the ablation process, the gelable hydrodissection material 22 may be bio absorbed and discharged from the body, or cooled and manually extracted.

### Example I

### Poloxamer-407 in Water

In a first embodiment, the gelable hydrodissection material 22, for example, described with respect to FIG. 1, may be a polymer such as a poloxamer and specifically Poloxamer-407.

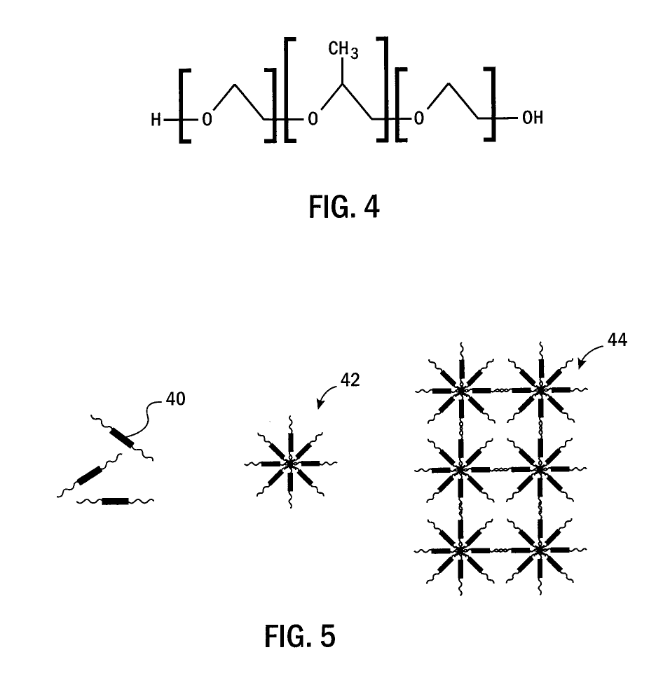

Referring now to FIG. 4, in a chemical formula for a solute of one hydrodissection material (e.g. Poloxamer-407) consisting of a hydrophobic center block flanked by two hydrophilic end blocks. Poloxamers are nonionic triblock copolymers having a central hydrophobic block of polypropylene oxide (PPO) or polypropylene glycol (PPG) flanked by two hydrophilic blocks of polyethylene oxide (PEO) or polyethylene glycol (PEG). The term “oxide” is used for high molar mass polymers whereas the term “glycol” is used for low to medium range molar mass polymers. The molecule has the general formula HO—(C₂H₄O)ₐ(C₃H₆O)_{b}(C₂H₄O)_{c}—H in which a, b, and c are integers and a and c are approximately equal. (C₃H₆O)_{b} represents the hydrophobic block, and (C₂H₄O)ₐ and (C₂H₄O)_{c} represent the hydrophilic blocks. A shorthand representation of poloxamer is HO-Pol-H or alternatively, is shown by the repeating triplet pattern PEO-PPO-PEO or PEG-PPG-PEG.

The hydrophobic base is created by adding propylene oxide to the two hydroxyl groups of a propylene glycol nucleus. The hydrophobic base can be made to any controlled length. By adding ethylene oxide to the hydrophobic base, it is possible to put polyethylene oxide hydrophilic groups on both ends of the molecule. The hydrophilic groups can also be controlled to constitute a given length. The lengths of the polymer blocks (i.e. degree of polymerization) can vary between various polymeric constructs. Because of this, numerous poloxamers exist with a wide range of unique properties.

Many poloxamers with different compositions and molecular weights are available commercially. Poloxamers are commonly named with the letter “P” followed by three digits. The first two digits×100 give the approximate molecular mass of the polypropylene oxide core, and the last digit×10 gives the percentage polyethylene oxide content. Poloxamers are freely soluble in water and in alcohol.

In one aspect of the present invention, the gelable hydrodissection material 22 is a Poloxamer-407 solution. Poloxamer-407 is a nonionic triblock copolymer with the approximate length of the two hydrophilic blocks being 101 repeat units (a, c=101) while the approximate length of the hydrophobic block being 56 repeat units (b=56). Poloxamer-407 is represented by the formula HO—(C₂H₄O)₁₀₁(C₃H₆O)₅₆(C₂H₄O)₁₀₁—H. Poloxamer-407 has an average molecular weight of 9840 to 14600, weight percent of polyethylene oxide of 73.2±1.7, and unsaturation of 0.048±0.017 mEq/g. The compound has the BASF trade name Lutrol F 127.

Solid Poloxamer-407 is readily water soluble and when mixed with water, forms a thermoreversible gel. With concentrations greater than 10 w/w % it changes to plastic flow with a pronounced change in flowability and viscosity. The Poloxamer-407 solution can be prepared by dissolving the polymer at temperatures exceeding 70 degrees Celsius or in the cold at around 5 to 10 degrees Celsius. The sol-gel transition temperature (i.e. gelation temperature) of Poloxamer-407 solutions range around 15 to 25 degrees Celsius at polymer concentrations greater than 16%.

Referring now to FIG. 5, Poloxamer-407 exhibits the unique characteristic of thermoreversibility, which occurs by micelle formation in an aqueous solution. As temperature increases, the hydrophobic blocks of the free molecules 40 become dehydrated and begin to clump together forming micelles 42. Eventually, more micelles 42 form and the free hydrophilic chains become entangled. This leads to a formation of an organized structure 44 of micelles 42, which causes a phase change to occur. This phase change occurs at the gelation temperature in which the liquid becomes a gel. The gelation temperature varies depending on the concentration of poloxamer in solution. Breakdown of the poloxamer occurs in the body as the solution becomes dilute and the formed micelles 42 are dismembered. The temperature at which the poloxamer begins to precipitate out of solution is the gel melting temperature. In vitro breakdown of the poloxamer depends on poloxamer concentration, temperature, and pH.

Poloxamers are considered bioabsorbable when the polymer has a molecular weight less than 13 kDa. As a bioabsorbable substance, poloxamer chains are absorbed into the blood stream and passed out of the body through the kidneys. The general process for this process consists of the poloxamer being diffused from the blood into the nephrons of the kidneys. Diffusion of sugars and water back into the blood occurs in the tubules, which eventually make the urine very concentrated. The poloxamer is passed through these tubules leading to the bladder and finally is excreted in the urine. This whole process is expected to take up to three days.

Poloxamers lack any inherent myotoxicity following single or multiple intramuscular injections. Toxicity is comparable to that of saline or peanut oil. Poloxamer-407 is well tolerated when administered subcutaneously. Poloxamer-407 is an inactive ingredient for inhalation, oral solutions, suspensions, ophthalmics, topical formulations, and IV injections. OSHA has classified it as non-hazardous. It exhibits a pH of 6.0-7.5 in aqueous solutions, which is similar to the human body.

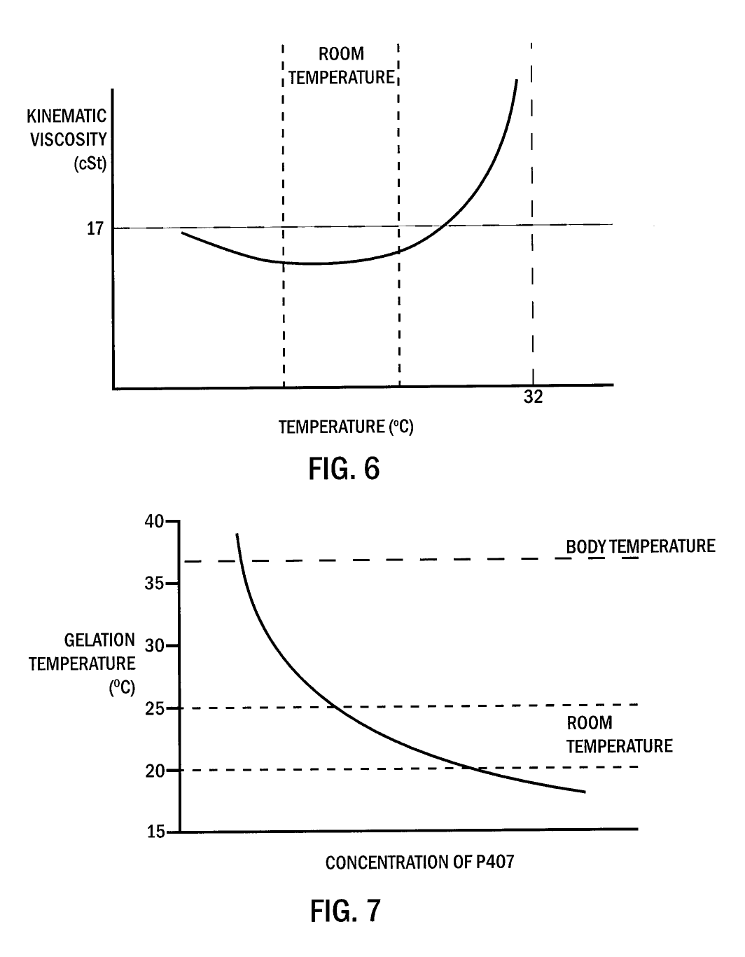

Referring now to FIG. 6, the solution of Poloxamer-407 used as the gelable hydrodissection material 22 may be adjusted to have a relatively low viscosity at room temperature (below 18 centiStokes) and thus to be introducible through a hypodermic needle but then to increase in viscosity at body temperature (above 18 centiStokes) to provide reduced migration. At high temperatures past the gel melting temperature, the viscosity again drops, however, it is believed that in such cases where the high temperature results from ablation, the liquefied gelable hydrodissection material 22 is minor and blocked from further movement by the remaining mass of gelable hydrodissection material 22 and in any case the material degrades to a state comparable to D5W.

Referring now to FIG. 7, adjustment of the gelation temperature can be done by changing the concentration of the Poloxamer-407 in water. A weight-based dilution ratio of between 14 and 18 percent Poloxamer-407 or roughly 15.4 percent has been determined to be acceptable.

Generally, Poloxamer-407 is iso-dense (for computed tomography) and iso-echoic (for ultrasound) compared to water and watery tissues, and thus difficult to discern from adjacent fluid filled structures such as bowel without the introduction of contrast agents 29 or bounding by other visible tissue.

### Example II

### Poloxamer-407 in Water with Benzoic Acid

Alternatively or in addition, benzoic acid may be used as an additive to decrease the viscosity of the Poloxamer-407 solution described above to facilitate injection. Benzoic acid is a common additive in many foods and oral solutions as a preservative, and included as an additive in medications administered topically, intravenously, intramuscularly, and rectally. It is categorized by the FDA as GRAS (Generally Recognized as Safe). A low concentration, 0.5-2.0 w/w % would be used for each unit (250 ml) of poloxamer solution. The addition of benzoic acid would allow for the concentration of Poloxamer-407 to be reduced while still maintaining the desired sol-gel transition temperature. The decrease in Poloxamer-407 concentration would lower the viscosity of the poloxamer solution, thus facilitating injection into the tissue.

### Example III

### Poloxamer-407 in Water with Poloxamer 188

In another aspect of the present invention, poloxamer 188 may be used as an additive to increase the gelation temperature of the Poloxamer-407 solution described in either example above. Poloxamer 188 is a triblock copolymer with 106 PEO blocks and 27 PPO blocks. It is nonionic, bioabsorbable, and has a molecular weight less than 13 kDa. Poloxamer 188 has lesser gelling qualities in concentrations greater than 20 w/w %, but still gels at concentrations less than 20 w/w %. As poloxamer 188 is added, the gelation temperature increases to a maximum, then decreases as more is added. Poloxamer 188 would be anticipated to increase bio-adhesion while also increasing viscosity, thus would likely be used in conjunction with a viscosity reducing additive.

### Example IV

### Poloxamer-407 in Water with Methylcellulose

In another aspect of the invention, methylcellulose (MC) may be added to the Poloxamer-407 solution described in any example above. Methylcellulose is a hydrophilic compound derived from cellulose, a polysaccharide consisting of many linked D-glucose units. Depending on the R groups attached to it, MC can be characterized as a variety of reagents, such as hypromellose (HPMC) or hydroxyethyl cellulose (HEC). These cellulose derivatives are non-toxic and non-allergenic, though not digestible. MC and its various forms have been used as thickeners and emulsifiers, constipation treatments, lubricants, glues/binders, foam stabilizers, dough strengtheners, and long-term drug release gels.

Use of MC as an additive would be anticipated to increase adhesion strength and reduce solution viscosity. MC imparts substantial mucoadhesive force to poloxamer solutions without damaging mucosa or submucosa. Additionally, MC has been shown to reduce the gelation temperature and increase the gel strength. These solutions only require 1-2 w/w % of MC. Methylcellulose may also form an alternative to the Poloxamer-407 as a standalone hybrid dissection material.

### Example V

### Poloxamer-407 in Water with Polyethylene Glycol

In another aspect of the invention, polyethylene glycol 400 (PEG 400) may be added to the Poloxamer-407 solution to decrease the viscosity of the solution. PEG 400 is a low molecular weight, highly hydrophilic polymer. Since the PEG 400 molecule is hydrophilic, it binds with free water molecules in the solution. With less free water molecules in solution, PEO chain entanglement occurs sooner and the gelation temperature is lower. The addition of PEG 400 also increases the elastic modulus of the poloxamer gel.

### Example VI

### Sodium Alginate

In a second embodiment, the gelable hydrodissection materials 22′ and 22″, for example, described with respect to FIG. 2 may be an alginate combined with multivalent cations, and specifically sodium alginate as material 22′ and mulivalent cations Ca²⁺ as material 22″.

Alginate is a block copolymer composed of homopolymeric regions of two monasaccharides 1,4-linked β-D-mannuronic acid (M) blocks and 1,4-linked α-L-guluronic acid (G) blocks, and interspersed with regions of alternating structure. The gelling properties of alginate depend on the ratio of the two M and G blocks as well as the blocks of MM, GG, and irregular M and G sequences, their block length and arrangement. Alginate exhibits a unique, almost temperature-independent sol-gel transition in the presence of multivalent cations (e.g., Ca²⁺). Alginates with high guluronic acid have enhanced ability to make gels because Ca²⁺ ions appear to bind in preference to G blocks. Alginates with more than 70% G blocks have the highest mechanical strength, porosity and stability towards monovalent cations as well as the lowest shrinkage. These qualities provide gel formation, viscosity, and stability.

Sodium alginate is a sodium salt of alginic acid. Its empirical formula is NaC₆H₇O₆. It is a GRAS substance derived from brown algae.

Hydrodissection material, as used herein, refers to a fluid material for separating tissues and is not intended to be limited to aqueous solutions. The invention contemplates use of this material generally to protect tissues from any destructive agent used to treat nearby tissues whether or not the process is technically termed ablation. It will be appreciated that other methods of controlling the solidification of the fluid material may be employed, for example, including control of pH, density, ion concentration, cellular interaction, etc.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

What we claim is:

1. A method of tumor treatment comprising the steps of:
(1) introducing a biocompatible gelable liquid in a liquid phase between a first and second tissue region to separate the regions;
(2) converting the liquid phase gel to a gel phase having a substantially greater viscosity to resist migration from between the first and second tissue regions; and
(3) applying a destructive agent to the first tissue to destroy a tumor portion thereof;
wherein the separation of the first and second tissue is selected to protect the second tissue from the destructive agent applied to the first tissue.

2. The method of claim 1 wherein the gelable liquid changes from the liquid phase to the gel phase as a function of temperature and wherein the gel phase occurs at substantially normal body temperature which causes a temperature induced phase change of the gelable liquid; and
wherein step (2) is provided by a temperature change of the gelable liquid.

3. The method of claim 2 wherein step (1) includes the step of cooling the gelable liquid to substantially no greater than room temperature at a time of introduction.

4. The method of claim 3 wherein the gelable liquid is a poloxamer.

5. The method of claim 1 wherein the biocompatible gelable liquid changes from the liquid phase to the gel phase in the presence of a gelling trigger material; and
wherein step (2) is provided by the introduction of the gelling trigger material into contact with the introduced gelable liquid.

6. The method of claim 5 wherein the gelable liquid is sodium alginate.

7. The method of claim 1 wherein the ablation is selected from the group consisting of: cryoablation, microwave ablation, radiofrequency ablation, laser ablation, ethanol ablation, chemoembolization, interstitial or external ultrasound ablation, internal or external radiotherapy.

8. The method of claim 1 wherein the first and second tissues are selected from tissue pair groups of: liver/diaphragm, liver/body wall, liver/bowel, liver/stomach, kidney/bowel, kidney/ureter, kidney/pancreas, kidney/psoas and ilioinguinal nerve, and gallbladder/liver.

9. The method of claim 1 wherein the gelable liquid includes a contrast agent and further including the step of monitoring the introduction of the contrast agent with an image modality sensitive to the contrast agent.

10. The method of claim 1 wherein the step of introducing the gelable liquid includes injecting the gelable liquid through a hypodermic needle having an inner diameter no greater than one millimeter.

11. The method of claim 1 wherein the step of introducing the gelable liquid creates a layer of gel of the liquid between the first and second tissue of greater than five millimeters.

12. A hydrodissection material for providing a barrier between two tissue regions, one subject to the application of a destructive agent, the hydrodissection material comprising a biocompatible gelable liquid having a gel phase viscosity of greater than 18 centiStokes at body temperature and a liquid phase viscosity of less than 18 centiStokes at room temperature to be introducible through a hypodermic needle between tissue regions to create barriers there between.

13. The hydrodissection material of claim 12 wherein the gelable liquid molecular weight is less than 13 KDa.

14. The hydrodissection material of claim 12 wherein the gelable liquid is a micelle forming polymer.

15. The hydrodissection material of claim 14 wherein the gelable liquid is a solution of a poloxamer.

16. The hydrodissection material of claim 15 wherein the hydrodissection material is a solution of Poloxamer-407 water having a weight-based dilution ratio of between 14 and 18 percent Poloxamer-407.

17. The hydrodissection material of claim 12 wherein the hydrodissection material further includes a contrast agent for a medical imaging modality.

18. The hydrodissection material of claim 12 wherein the hydrodissection material further includes a contrast agent selected from the group of: ultrasound blocking microspheres, ultrasound blocking isohexyl, x-ray blocking iodine, and MRI sensitive gadolinium.

19. The hydrodissection material of claim 12 wherein the hydrodissection material further includes one half to three percent weight to volume of isohexyl.

20. The hydrodissection material of claim 12 wherein the hydrodissection material further includes an additive selected from the group consisting of polyethylene glycol, methyl cellulose, Poloxamer 188, and benzoate acid.
